(19) United States
(12) Patent Application Publication
(10) Pub. No.: US 2019/0167587 A1
(43) Pub. Date: Jun. 6, 2019

(54) PHARMACEUTICAL COMPOSITION FOR PREVENTION OR TREATMENT OF PULMONARY DISEASE INCLUDING MESENCHYMAL STEM CELL-DERIVED ARTIFICIAL NANOSOMES

(71) Applicant: MDIMUNE INC., Seoul (KR)

(72) Inventors: Shin Gyu BAE, Seongnam-si (KR); Yeon-Mok OH, Seoul (KR)

(73) Assignee: MDIMUNE INC., Seoul (KR)

(21) Appl. No.: 16/205,277

(22) Filed: Nov. 30, 2018

(30) Foreign Application Priority Data
Dec. 1, 2017 (KR) 10-2017-0163897

(51) Int. Cl. A61K 9/127; A61K 35/28; A61P 11/00

(52) U.S. Cl. CPC A61K 9/1271; A61P 11/00; A61K 35/28; A61K 9/1277

(57) ABSTRACT

Provided herein is a method for treating a pulmonary disease using mesenchymal stem cell-derived artificial nanosomes exhibiting a significantly excellent capability to regenerate alveoli compared to mesenchymal stem cells themselves and natural exosomes derived from human adipose-derived mesenchymal stem cells, wherein the mesenchymal stem cell-derived artificial nanosomes of the present disclosure may be collected through a simple production method using an extruder and an ultracentrifuge, instead of treating mesenchymal stem cells with a separate chemical substance and the potential side effects caused by stem cell therapeutic agents is low.

### CROSS-REFERENCE TO RELATED APPLICATION

This application claims priority to and the benefit of Korean Patent Application No. 10-2017-0163897, filed on Dec. 1, 2017, the disclosure of which is incorporated herein by reference in its entirety.

### BACKGROUND

### 1. Field of the Invention

The present disclosure relates to a pharmaceutical composition for the prevention or treatment of a pulmonary disease, which includes mesenchymal stem cell-derived artificial nanosomes.

### 2. Discussion of Related Art

Chronic pulmonary diseases are known to progress such that pulmonary inflammation occurs due to various causes and lungs are gradually destroyed. A considerable number of acute or chronic respiratory diseases such as asthma, chronic obstructive pulmonary disease (COPD), pulmonary fibrosis, and the like have conditions with inflammation in the airway and pulmonary parenchyma. Infiltrated inflammatory cells cause damage to bronchial and pulmonary tissues, which eventually results in respiratory dysfunctions characteristic of the above diseases, such as reduction in respiratory flow rates or oxygen exchange capacity. Thus, drugs exhibiting an anti-inflammatory effect have been applied to the treatment of inflammatory respiratory diseases, and it has been known that adrenocortical steroids are effective in mild to moderate bronchial asthma. In addition, adrenocortical steroids have been reported to prevent exacerbation of COPD, but the effects thereof on the condition of COPD are limited, and the efficacy of adrenocortical steroids in pulmonary fibrosis has not yet been distinctly verified. Meanwhile, adrenocortical steroids have been known not only to non-specifically inhibit immune functions, but also to possibly cause various side effects such as electrolyte abnormality, peptic ulcer, myopathy, behavioral abnormality, cataract, osteoporosis, osteonecrosis, growth inhibition, and the like. With the pervasion of therapies mainly using an inhaled steroid for the treatment of bronchial asthma, the number of emergency outpatients or inpatients with asthmatic attack has decreased, and the number of controllable outpatients has increased. Even under such circumstances, the number of patients is not reduced, and asthmatic deaths due to lethal seizures still occur, and thus currently available anti-asthmatic combination therapies mainly using an inhaled steroid still do not exhibit satisfactory therapeutic effects. Therefore, there is a need to develop a new therapeutic agent having high efficacy and reduced side effects.

Meanwhile, emphysema refers to abnormal and permanent peripheral airway and alveolar expansions due to the destruction of the distal airspace of terminal bronchioles. The long-term emphysema causes considerable damage to the alveoli, which are small alveolar sacs that exchange oxygen and carbon dioxide in the lungs, and capillaries, which leads to pulmonary hypertension, especially secondary pulmonary hypertension. Although several substances for the prevention and treatment of emphysema and pulmonary hypertension are known, the types thereof are limited and the therapeutic effects thereof are also insignificant, and thus research is needed to develop more effective drugs. Recent studies have reported that emphysema is associated with elastase, and neutrophil elastase (or white blood cell elastase) is a serine protease belonging to the same family as chymotrypsin, and exhibits various substrate specificities. Neutrophil elastase has a charge transfer system composed of a catalytic triad consisting of histidine, aspartate, and serine residues along with other serine proteases, and these histidine, aspartate, and serine residues are dispersed in the primary sequence of a polypeptide, while gathering together in proteins folded into a tertiary structure. Neutrophil elastase is a powerful nonspecific serine protease that decomposes elastin, which exhibits physical characteristics of connective tissues by composing elastic fibers together with collagen, and acts as a bactericidal substance when the immune system is degraded by an intraphagosomal process, and accelerates inflammation, emphysema, and chronic obstructive disease. Known causes of the development of emphysema include combinations of inflammation, elastase, matrix metalloprotease imbalance, auto-apoptosis, and oxidative stress. Especially, when the lungs are exposed to porcine pancreatic elastase which is an enzyme with excellent activity against elastin, neutrophils and macrophages have been shown to induce acute pulmonary inflammatory responses. In addition, the production of reactive oxygen species (ROS) is induced by neutrophil elastase, and research results have shown that increases in an MUC5AC messenger RNA level in neutrophil elastase are dependent on the generation of an intracellular oxidant or changes in a cell redox state, which indicates that ROS plays a certain role in elastase-mediated inflammation. Nitrogen oxide is also known to play a major role in elastase-mediated diseases.

As described above, there is a need to develop a novel therapeutic agent for the treatment of a variety of pulmonary diseases including emphysema with excellent efficiency, and thus research thereon continues to be conducted (see Korean Patent Application Registration No. 10-1189655).

### SUMMARY OF THE INVENTION

As a result of having made intensive efforts to develop a substance having reduced side effects and excellent preventive and therapeutic effects against pulmonary diseases occurring by destruction of the lungs due to various causes, including emphysema, the inventors of the present disclosure verified that in particular, mesenchymal stem cell-derived artificial nanosomes had an excellent effect of inducing regeneration of alveoli, thus completing the present disclosure.

Provided is a pharmaceutical composition for preventing or treating a pulmonary disease, which includes mesenchymal stem cell-derived artificial nanosomes.

Provided is a cell therapeutic composition for preventing or treating a pulmonary disease, which includes mesenchymal stem cell-derived artificial nanosomes.

Additional aspects will be set forth in part in the description which follows and, in part, will be apparent from the description, or may be learned by practice of the presented embodiments.

According to an aspect of the present disclosure, there is provided a pharmaceutical composition for preventing or treating a pulmonary disease, which includes artificial nanosomes derived from mesenchymal stem cells.

In one embodiment of the present disclosure, the artificial nanosomes derived from mesenchymal stem cells may induce regeneration of alveoli.

In another embodiment of the present disclosure, the mesenchymal stem cells may be derived from umbilical cord, umbilical cord blood, bone marrow, muscle, nerves, skin, amniotic membrane, placenta, or fat, but the present disclosure is not limited thereto. The mesenchymal stem cells may be derived from human fat.

In another embodiment of the present disclosure, the artificial nanosomes derived from mesenchymal stem cells may be produced using a method selected from the group consisting of, but not being limited to, extrusion, sonication, cell lysis, homogenization, freezing-thawing, electroporation, mechanical degradation, and chemical treatment, and may be produced using, for example, an extruder and an ultracentrifuge.

In another embodiment of the present disclosure, the pulmonary disease may be at least one selected from the group consisting of emphysema, asthma, pneumonia, tuberculosis, pulmonary hypertension, lung cancer, bronchopulmonary dysplasia, chronic obstructive pulmonary disease, acute bronchitis, chronic bronchitis, bronchiolitis, bronchiectasis, hypersensitivity, pneumonitis, acute smoke inhalation, heat-induced lung injury, cystic fibrosis, pulmonary alveolar proteinosis, alpha-1-protease deficiency, pulmonary inflammatory disorder, acute respiratory distress syndrome, acute lung injury, idiopathic respiratory distress syndrome, and idiopathic pulmonary fibrosis, but the present disclosure is not limited thereto. In addition, when the pulmonary disease is emphysema, the emphysema may be at least one selected from the group consisting of panlobular emphysema, centrilobular emphysema, interstitial emphysema, and compensatory emphysema.

In another embodiment of the present disclosure, the artificial nanosomes derived from mesenchymal stem cells increase the expression of a growth factor, wherein the growth factor may be, but is not limited to, for example, at least one selected from the group consisting of vascular endothelial growth factor (VEGF), fibroblast growth factor 2 (FGF2), and hepatocyte growth factor (HGF).

The present disclosure also provides a cell therapeutic composition for preventing or treating a pulmonary disease, which includes mesenchymal stem cell-derived artificial nanosomes.

The present disclosure also provides a method of preventing or treating a pulmonary disease, including administering a pharmaceutical composition for preventing or treating a pulmonary disease, which includes mesenchymal stem cell-derived artificial nanosomes, to an individual.

The present disclosure also provides a use of mesenchymal stem cell-derived artificial nanosomes for the prevention or treatment of a pulmonary disease.

### BRIEF DESCRIPTION OF THE DRAWINGS

The above and other objects, features and advantages of the present disclosure will become more apparent to those of ordinary skill in the art by describing in detail exemplary embodiments thereof with reference to the accompanying drawings, in which:

FIG. 1A is a transmission electron microscope (TEM) image of artificial nanosomes derived from human adipose-derived mesenchymal stem cells;

FIG. 1B illustrates images showing results of verifying the expression of CD63, CD9, and CD81, which are exosome markers, by western blotting;

FIG. 1C illustrates graphs showing the size distribution of artificial nanosomes derived from human adipose-derived mesenchymal stem cells and the size distribution of natural exosomes derived from human adipose-derived mesenchymal stem cells;

FIG. 2 is a graph comparatively showing degrees of cell growth when treated with artificial nanosomes derived from human adipose-derived mesenchymal stem cells (Nano) and natural exosomes derived from human adipose-derived mesenchymal stem cells (Exo);

FIG. 3A illustrates images comparatively showing capabilities to regenerate alveoli of artificial nanosomes derived from human adipose-derived mesenchymal stem cells, natural exosomes derived from human adipose-derived mesenchymal stem cells, and mesenchymal stem cells;

FIG. 3B is a graph showing mean linear intercept (MLI) results of the capabilities to regenerate alveoli of artificial nanosomes derived from human adipose-derived mesenchymal stem cells, natural exosomes derived from human adipose-derived mesenchymal stem cells, and mesenchymal stem cells;

FIG. 4 illustrates graphs showing results of verifying changes in expression amounts of vascular endothelial growth factor (VEGF), fibroblast growth factor 2 (FGF2), and hepatocyte growth factor (HGF) when treated with each of artificial nanosomes derived from human adipose-derived mesenchymal stem cells, natural exosomes derived from human adipose-derived mesenchymal stem cells, and mesenchymal stem cells;

FIG. 5 illustrates images comparatively showing capabilities to regenerate alveoli of artificial nanosomes derived from umbilical cord-derived mesenchymal stem cells and mesenchymal stem cells;

FIG. 6 is a graph showing MLI results of the capabilities to regenerate alveoli of artificial nanosomes derived from umbilical cord-derived mesenchymal stem cells and mesenchymal stem cells; and

FIG. 7 illustrates results of verifying changes in expression amounts of VEGF, FGF2, and HGF when treated with each of artificial nanosomes derived from umbilical cord-derived mesenchymal stem cells and mesenchymal stem cells.

### DETAILED DESCRIPTION OF EXEMPLARY EMBODIMENTS

Reference will now be made in detail to embodiments, examples of which are illustrated in the accompanying drawings, wherein like reference numerals refer to like elements throughout. In this regard, the present embodiments may have different forms and should not be construed as being limited to the descriptions set forth herein. Accordingly, the embodiments are merely described below, by referring to the figures, to explain aspects.

As a result of having made intensive efforts to develop a substance having an excellent preventive or therapeutic effect in various pulmonary diseases including emphysema, the inventors of the present disclosure produced mesenchymal stem cell-derived artificial nanosomes by using an extruder and an ultracentrifuge, and verified that the artificial nanosomes could more effectively induce the generation of alveoli in damaged pulmonary tissue than mesenchymal stem cells, thus completing the present disclosure.

Hereinafter, the present disclosure will be described in detail.

The present disclosure provides a pharmaceutical composition for the prevention or treatment of a pulmonary disease, which includes artificial nanosomes derived from mesenchymal stem cells.

As used herein, the term “mesenchymal stem cells” is referred to as MSCs as an abbreviation therefor. The mesenchymal stem cells are one type of stem cells collected from bone marrow, umbilical cord blood, and adipose tissue, are known to exist in the body in a number of approximately 1 million, have a fibroblast shape, are able to grow indefinitely in the laboratory, and may differentiate into important cell types such as adipocytes, osteoblasts, and chondrocytes, unlike blood stem cells.

In addition, the term “nanosomes” as used herein refers to nanoscale liposomes that have a microscale spherical shape and may be used to send specific active ingredients into the tissue.

In addition, as used herein, the term “chronic obstructive pulmonary disease (COPD)” refers to a respiratory disease occurring such that abnormal inflammation reaction occurs in the lungs due to inhalation of harmful particles such as smoking or the like or inhalation of industrial gases, which results in progressive airflow limitation, thus degrading pulmonary functions and causing dyspnea. More particularly, when the airway and bronchus are repeatedly irritated with an airway irritating substance such as tobacco smoke, the secretion of bronchial mucus, which is secreted to remove a foreign substance in the bronchus, increases, which results in partial or complete obstruction of the bronchus. When the bronchus is obstructed, the alveoli expand and become damaged, and thus impairing an ability to exchange oxygen and carbon dioxide, and the obstructed alveoli are easily infected by bacteria. When a COPD patient is infected by bacteria, arterial blood oxygen pressure is rapidly reduced due to severe gas exchange disorder, and thus, when arterial blood carbon dioxide pressure is increased by respiratory failure, carbon dioxide narcosis may occur. Non-limiting examples of the COPD include, but are not limited to, emphysema and chronic bronchitis.

The term “prevention” as used herein means all actions that inhibit COPD or delay the onset thereof via administration of the pharmaceutical composition according to the present disclosure.

In addition, the term “treatment” as used herein means all actions that alleviate or beneficially change symptoms due to COPD via administration of the pharmaceutical composition according to the present disclosure.

In one embodiment of the present disclosure, to produce artificial nanosomes derived from human adipose-derived mesenchymal stem cells (ADSCs), ADSCs were cultured, and then artificial nanosomes were isolated therefrom using an extruder and an ultracentrifuge (see Example 1).

In another embodiment of the present disclosure, a mouse model with emphysema induced by an elastase treatment was constructed (see Example 2), and capabilities to regenerate alveoli of the ADSC-derived artificial nanosomes produced using the method of Example 1 and natural exosomes were compared with each other (see Example 3).

In another embodiment of the present disclosure, by treating damaged pulmonary tissues of the emphysema animal model constructed using the method of Example 2 with ADSC-derived artificial nanosomes and natural exosomes, changes in expression amounts of vascular endothelial growth factor (VEGF), fibroblast growth factor 2 (FGF2), and hepatocyte growth factor (HGF), which are growth factors, were identified (see Example 4).

In another embodiment of the present disclosure, artificial nanosomes derived from umbilical cord-derived mesenchymal stem cells were produced (see Example 5), and by treating damaged pulmonary tissues of the emphysema animal model constructed using the method of Example 2 with the artificial nanosomes derived from umbilical cord-derived mesenchymal stem cells and mesenchymal stem cells, the capabilities thereof to regenerate alveoli were compared with each other (see Example 6), and changes in expression amounts of VEGF, FGF2, and HGF were identified (see Example 7).

The pharmaceutical composition of the present disclosure may be administered via any general administration route as long as it can reach the target tissue. That is, the pharmaceutical composition may be administered orally, intraperitoneally, intravenously, intramuscularly, subcutaneously, intradermally, intranasally, intrapulmonarily, intrarectally, intradurally, or intrapleurally, but the present disclosure is not limited thereto. For example, the pharmaceutical composition may be administered intrapulmonarily or intrapleurally for the treatment of a pulmonary disease.

In addition, the pharmaceutical composition according to the present disclosure may further include a pharmaceutically acceptable carrier, and the pharmaceutically acceptable carrier may include any carrier commonly used for preparations, such as saline, sterilized water, Ringer's solution, buffered saline, cyclodextrin, dextrose solution, maltodextrin solution, glycerol, ethanol, and liposome, but the present disclosure is not limited thereto. According to need, the pharmaceutical composition may further include other general additives such as an antioxidant, buffer, and the like. In addition, the pharmaceutical composition may be formulated into injectable preparations such as an aqueous solution, a suspension, an emulsion, or the like; pills; capsules; granules; or tablets by further adding a diluent, a dispersant, a surfactant, a binder, a lubricant, or the like thereto. With regards to suitable pharmaceutically acceptable carriers and formulation, preparations may be formulated according to each ingredient by using a method disclosed in the Remington's reference (Remington' Pharmaceutical Science, Mack Publishing Company, Easton Pa.).

In addition, the composition according to the present invention is administered in a pharmaceutically effective amount. The term “pharmaceutically effective amount” as used herein refers to an amount sufficient to treat diseases at a reasonable benefit/risk ratio applicable to medical treatment, and an effective dosage level may be determined according to factors including the type of diseases of patients, the severity of disease, the activity of drugs, sensitivity to drugs, administration time, administration route, excretion rate, treatment period, and simultaneously used drugs, and factors well known in the medical field. The composition according to the present disclosure may be administered as an individual therapeutic agent or in combination with other therapeutic agents, may be administered consecutively or simultaneously with existing therapeutic agents, and may be administered in a single dose or multiple doses. It is important to administer the composition in the minimum amount that enables achievement of the maximum effects without side effects in consideration of all the above-described factors, and this may be easily determined by those of ordinary skill in the art.

In particular, an effective amount of the composition according to the present invention may vary according to ages, gender, and body weights of patients. Generally, the pharmaceutical composition may be administered in an amount of 0.001 mg to 150 mg, for example, 0.01 mg to 100 mg, per body weight (1 kg) daily or every other day, or may be administered once or three times a day. However, the dosage may be increased or decreased according to administration route, the severity of obesity, gender, body weight, age, and the like, and thus the dosage is not intended to limit the scope of the present invention in any way.

Another embodiment of the present disclosure provides a method of preventing or treating a pulmonary disease, the method including administering the composition to an individual.

The term “individual” as used herein refers to a subject with diseases requiring a treatment and, more particularly, includes mammals such as humans or non-human primates, e.g., mice, rats, dogs, cats, horses, cows, and the like.

Another embodiment of the present invention provides a use of mesenchymal stem cell-derived artificial nanosomes for the prevention or treatment of a pulmonary disease.

Hereinafter, exemplary embodiments will be described to aid in understanding of the present invention. However, the following examples are provided to more easily understand the present disclosure and are not intended to limit the scope of the present invention.

### Example 1. Production of Adipose Mesenchymal Stem Cell-Derived Artificial Nanosomes and Comparison Thereof with Natural Exosomes

1-1. Production of Adipose Mesenchymal Stem Cell-Derived Artificial Nanosomes

To produce human adipose-derived mesenchymal stem cells (ADSCs), a collected adipose tissue was washed with a buffer solution, and then contaminants such as red blood cells, white blood cells, and the like were removed therefrom, and the resulting tissue was cut into small sections and treated with collagenase II to decompose connective tissues. The suspended tissue was centrifuged and the supernatant was discarded, and the stromal vascular fraction (SVF), which is a precipitated layer, was filtered with a 100 μm nylon mesh (BD falcon) to remove a cell substrate that was not treated with the enzyme. The resulting SVF was centrifuged again to obtain a cell precipitate and the cell precipitate was cultured in a MesenPRO RS™ medium supplemented with growth supplement (Invitrogen) and 1% penicillin in a 5% CO₂ incubator at 37□, thereby obtaining adipose-derived mesenchymal stem cells.

7×10⁷ of the adipose-derived mesenchymal stem cells were resuspended in phosphate buffered saline (PBS), allowed to pass through 10 μm, 5 μm and 1 μm membranes using an extruder, and then artificial nanosomes were isolated therefrom using an ultracentrifuge. At this time, after primary ultracentrifugation with 40% and 10% opti-prep density gradient solutions, only a middle layer was separated and ultracentrifugation was performed thereon at 100,000 g force for 1 hour to collect pellets, the collected pellets were resuspended in PBS, and characteristics of the produced artificial nanosomes were evaluated and identified by a transmission electron microscope (TEM) and dynamic light scattering (DLS).

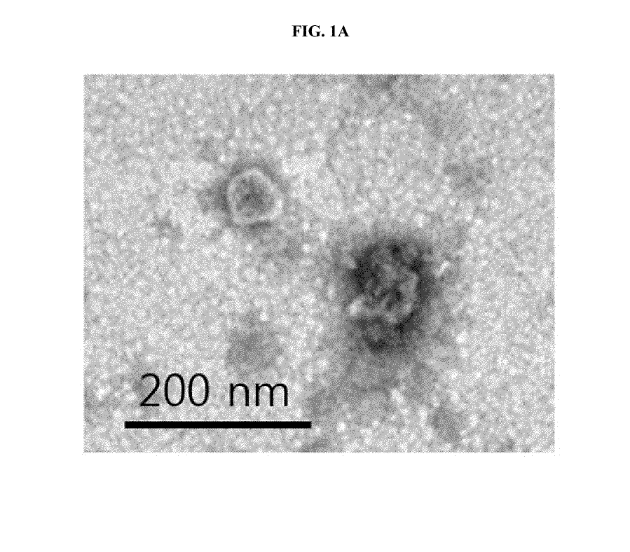

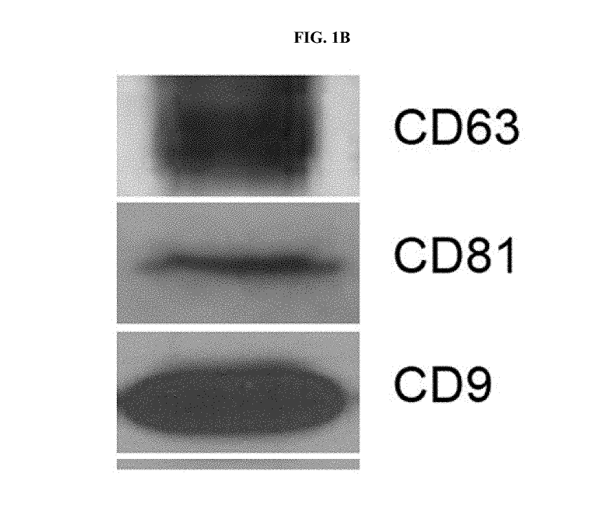

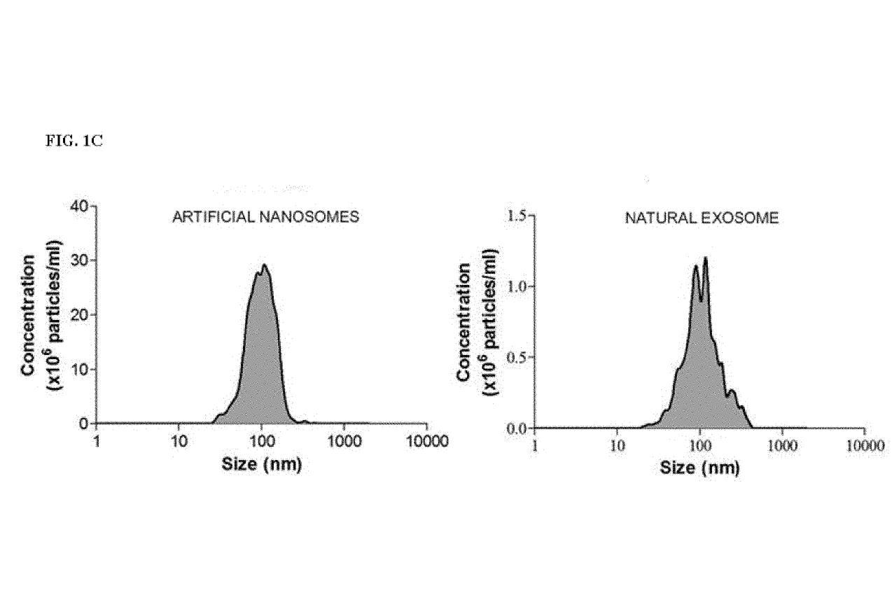

As a result, as illustrated in FIG. 1A, a TEM image of the artificial nanosomes was acquired, and, as illustrated in FIG. 1B, the expressions of CD63, CD9, and CD81, which are exosome markers, were verified by western blotting. The size distribution of the artificial nanosomes and natural exosomes through DLS is illustrated in FIG. 1C.

1-2. Comparison Between Artificial Nanosomes and Natural Exosomes

To compare a degree of cell growth of the artificial nanosomes derived from human adipose-derived mesenchymal stem cells (produced using the method of Example 1-1) with that of natural exosomes derived from human adipose-derived mesenchymal stem cells, a cell counting kit-8 (CCK 8) assay was conducted.

To collect natural exosomes derived from human adipose-derived mesenchymal stem cells, when a culture dish was filled with about 60% of human adipose-derived mesenchymal stem cells, a culture solution of the culture dish was exchanged with a culture medium containing 10% exosome depleted FBS, followed by culturing for 48 hours, to collect a culture solution. The culture solution itself was centrifuged at 300 g force for 10 minutes, at 200 g force for 10 minutes, at 10,000 g force for 30 minutes, and at 100,000 g force for 70 minutes, was washed with PBS to remove extra impurities, and then was centrifuged again at 100,000 g force for 70 minutes to collect natural exosomes.

0.1 μg/ml of MLE-12 cells (mouse lung epithelial cell line), which are alveolar epithelial cells (AECs), was treated with each of the natural exosomes collected using the above method and the artificial nanosomes produced using the method of Example 1-1 for 24 hours, and degrees of cell growth thereof were evaluated.

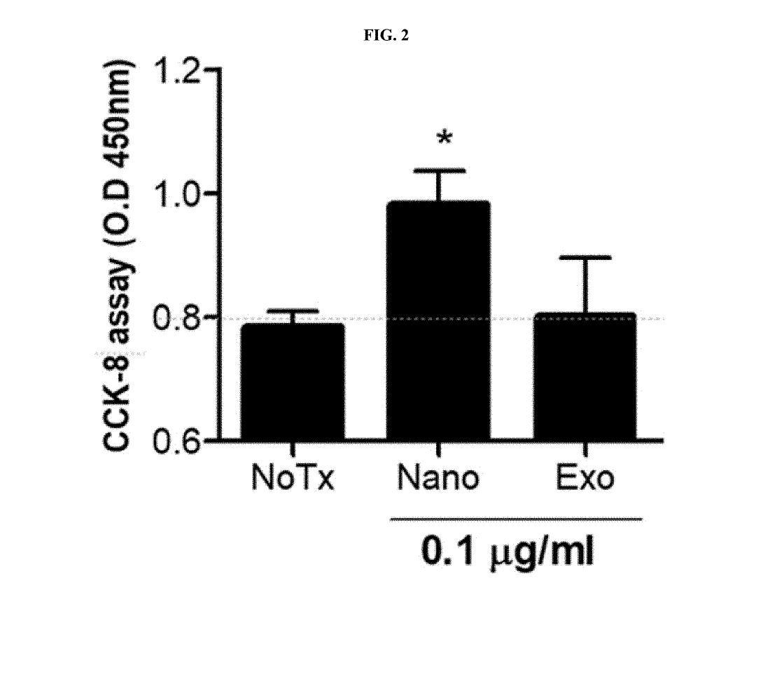

As a result, as illustrated in FIG. 2, it was confirmed that the artificial nanosome-treated cells (Nano) had a cell growth rate higher than that of the natural exosome-treated cells (Exo) by 20% or more, and the cell growth rate of the natural exosome-treated cells showed little difference from that of not treated cells (NoTx) as a control.

### Example 2. Production of Emphysema Animal Model

6-week-old female C57BL/6 mice having a weight of 20 g were purchased from Orient Bio, and then used in an experiment after one week of an inspection period.

To obtain an emphysema animal model, a disease animal model was produced by directly administering elastase to the airway of each mouse. In particular, the C57BL/6 mice were injected intraperitoneally with an anesthetic for injection and the upper teeth of the anesthetized mice were hung on a bar and fixed to straighten the airway. Thereafter, the mouth of each mouse was opened and the tongue was fixed to one side, and then the light for dissection was illuminated on the neck side to confirm that the light was coming in the empty space of the airway, and 0.5 unit/50 μl of elastase was injected into the light passage through the mouth of each mouse using a long tip (airway administration).

As described above, after administering elastase, the administered elastase was allowed to spread evenly to the lungs by shaking the corresponding mice leftward and rightward and forward and backward, and it was checked that the mice were awakened from the anesthesia. On day 7 after the airway administration of elastase, the mesenchymal stem cell-derived artificial nanosomes produced using the method of Example 1-1 were injected into the mice, and after one more week, the corresponding mice were sacrificed and the lungs were extracted therefrom to conduct an experiment for verifying the capability thereof to regenerate alveoli.

### Example 3. Verification of Capability of Adipose Mesenchymal Stem Cell-Derived Artificial Nanosomes to Regenerate Alveoli

To compare capabilities to regenerate alveoli with one another after being treated with each of artificial nanosomes derived from human adipose-derived mesenchymal stem cells, natural exosomes derived from human adipose-derived mesenchymal stem cells, and mesenchymal stem cells, mice, which were the elastase-induced emphysema animal model produced using the method of Example 2, were sacrificed, the lungs were extracted from the sacrificed mice, 0.5% low-melting agarose was inserted thereinto using a catheter to allow the alveoli to spread well, a pulmonary tissue was fixed using 4% formalin, and then H&E staining was performed through a paraffin embedment process, followed by observation using a microscope. At this time, to conduct comparison, “(−)” group into which elastase was not injected was used as a control, experimental groups were divided into “Ela” group into which only 0.4 U of elastase was injected, “Ela+ADSC” group treated with both elastase and 1×10⁵ mesenchymal stem cells, “Ela+Nano” group treated with both elastase and artificial nanosomes derived from human adipose-derived mesenchymal stem cells, and “Ela+Exo” group treated with both elastase and natural exosomes derived from human adipose-derived mesenchymal stem cells, and H&E staining was performed thereon.

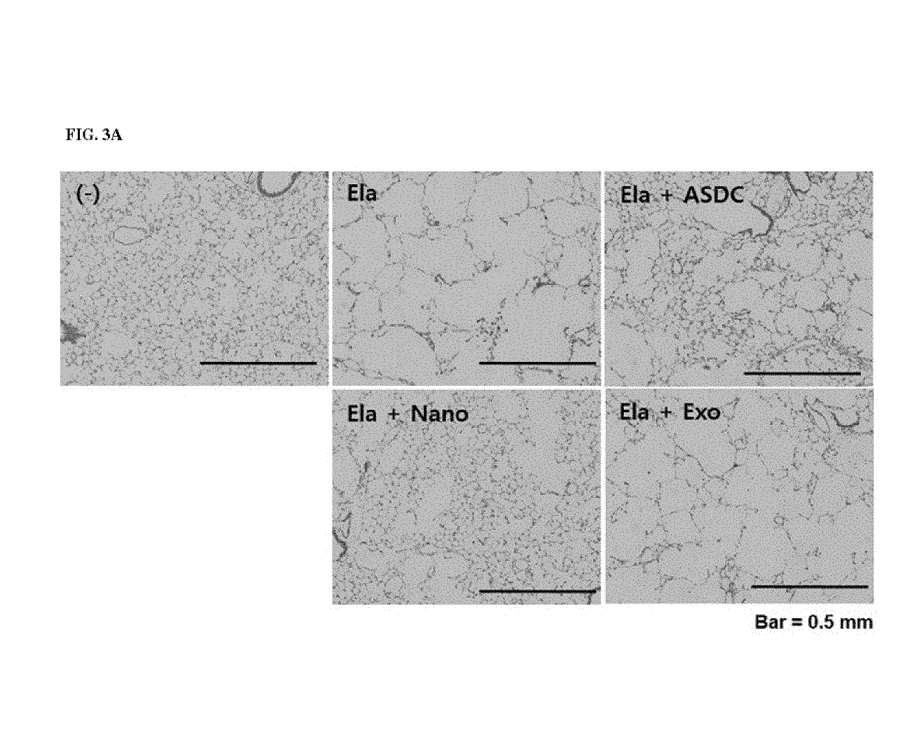

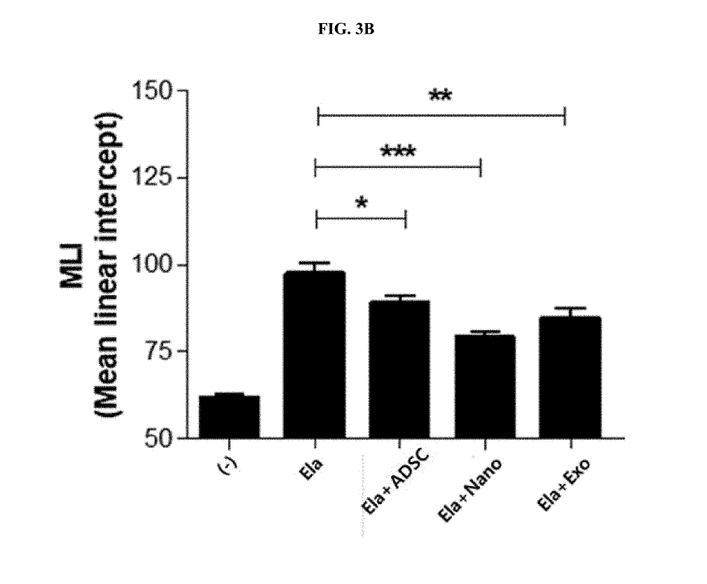

As a result of identifying a degree of alveolar regeneration by performing H&E staining on a pulmonary tissue, as illustrated in FIG. 3A, it was confirmed that the degree of H&E staining was low in the pulmonary tissues of the “Ela+ADSC” group and the “Ela+Exo” group due to poor regeneration, while the degree of H&E staining was high in the “Ela+Nano” group due to regeneration of the pulmonary tissue. From the results, it was confirmed that the “Ela+Nano” group exhibited a more excellent alveolar regeneration effect than that of the other groups.

In addition, results obtained by performing mean linear intercept (MLI) on the experimental results of FIG. 3A are illustrated in FIG. 3B.

In particular, a change in pulmonary tissues of the mouse model with emphysema induced by elastase treatment using the method of Example 2 was measured using MLI values from which a degree of destruction of alveoli was obtained, and MLI measurement was conducted by acquiring microscopic images of five areas per slide of the H&E-stained pulmonary tissue, drawing four lines corresponding to 1.0 mm bar for each image, and then calculating the number of alveoli within the lines. After calculating the number of alveoli per each image, the number of alveoli per mouse individual was averaged and the average number of alveoli was divided by 1.0 mm to obtain MLI values, and the MLI values were determined by two researchers under a blinded condition.

As a result, as illustrated in FIG. 3B, it was seen that, when MLI values, from which an alveolar regeneration rate was obtained, were measured, the “Ela” group treated only with elastase exhibited an increased MLI value compared to the “(−)” group as a control, which indicates a higher degree of destruction of alveoli, and it was confirmed that the “Ela+Nano” group exhibited a more excellent alveolar regeneration effect than that of the “Ela+ADSC” group and the “Ela+Exo” group.

From the above results, it can be seen that artificial nanosomes derived from adipose-derived mesenchymal stem cells exhibit an excellent alveolar regeneration effect.

### Example 4. Verification of Increase in Expression of Growth Factors of Adipose Mesenchymal Stem Cell-Derived Artificial Nanosomes

To measure changes in expression amounts of vascular endothelial growth factor (VEGF), fibroblast growth factor 2 (FGF2), and hepatocyte growth factor (HGF) when a damaged pulmonary tissue of the elastase-induced emphysema animal model produced using the method of Example 2 was treated with each of artificial nanosomes derived from human adipose-derived mesenchymal stem cells, natural exosomes, and mesenchymal stem cells, radioimmunoprecipitation assay (RIPA) buffer (Cell Signaling Technology, Danvers, Mass.) was added to 10 mg of a pulmonary tissue extracted from the animal model, followed by homogenization, and protein quantitation was performed thereon using the Brad-ford assay. At this time, VEGF, FGF-2, and HGF levels were measured by enzyme-linked immunosorbent assay (ELISA) according to the protocol of R&D systems (Minneapolis; MN).

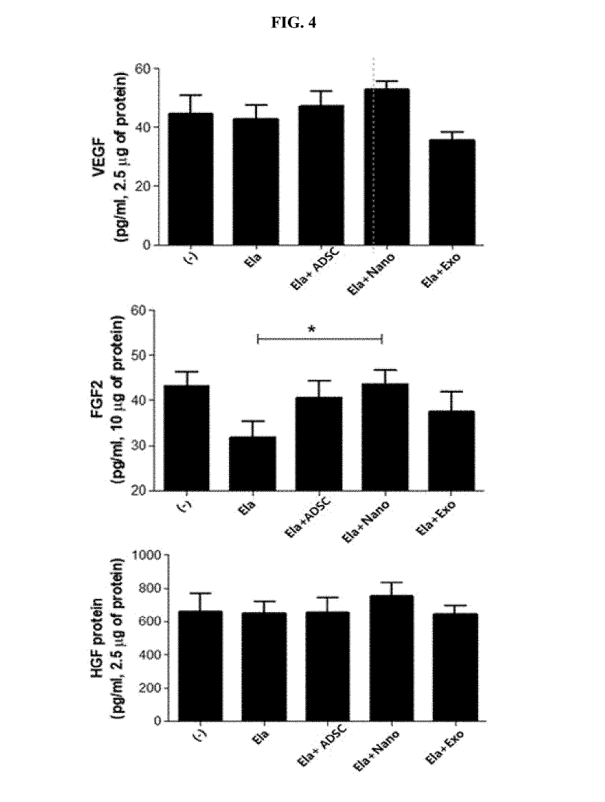

As a result, as illustrated in FIG. 4, it was confirmed that expression amounts of VEGF, FGF2, and HGF were significantly increased in the “Ela+Nano” group treated with both elastase and mesenchymal stem cell-derived artificial nanosomes compared to the “Ela+Exo” group treated with both elastase and mesenchymal stem cell-derived natural exosomes.

This indicates that the artificial nanosomes derived from adipose-derived mesenchymal stem cells have a potential capable of effectively inducing the regeneration of alveoli destructed by emphysema.

### Example 5. Production of Umbilical Cord Mesenchymal Stem Cell-derived Artificial Nanosomes

To produce human umbilical cord-derived mesenchymal stem cells (MSCs), arteries, veins, and umbilical membranes were removed from the umbilical cord of a human fetus, the tissue was cut into small sections, and then the tissue sections were treated with collagenase to decompose connective tissues. Subsequently, the resulting tissue sections were centrifuged after adding Dulbecco's Phosphate-Buffered Saline (DPBS) thereto, the supernatant was removed therefrom, the resultant was washed twice with low-glucose Dulbecco's Modified Eagle Medium (DMEM-LG Gibco), and the separated cells were cultured in DMEM-LG containing 3.7 mg/mL sodium bicarbonate and 10% FBS in a medium supplemented with 1% penicillin in a 5% CO₂ incubator at 37□, thereby obtaining umbilical cord-derived MSCs.

7×10⁷ of the MSCs were resuspended in PBS, allowed to pass through 10 μm, 5 μm, and 1 μm membranes using an extruder, and then artificial nanosomes were isolated therefrom using an ultracentrifuge. At this time, after primary ultracentrifugation with 40% and 10% opti-prep density gradient solutions, only a middle layer was separated and ultracentrifugation was performed thereon at 100,000 g force for 1 hour to collect pellets, the collected pellets were resuspended in PBS, and characteristics of the artificial nanosomes were evaluated and identified by DLS and a TEM.

### Example 6. Verification of Capability of Umbilical Cord MSC-Derived Artificial Nanosomes to Regenerate Alveoli

To compare capabilities to regenerate alveoli with each other after being treated with each of artificial nanosomes derived from human umbilical cord-derived mesenchymal stem cells and mesenchymal stem cells, mice, which were the elastase-induced emphysema animal model produced using the method of Example 2, were sacrificed, the lungs were extracted from the sacrificed mice, 0.5% low-melting agarose was inserted thereinto using a catheter to allow the alveoli to spread well, pulmonary tissue was fixed using 4% formalin, and then H&E staining was performed through a paraffin embedment process, followed by observation using a microscope. At this time, to conduct comparison, “Normal” group into which elastase was not injected was used as a control, and experimental groups were divided into “Elastase” group into which only elastase was injected, “MSCs” group treated with both elastase and mesenchymal stem cells, and “NVs (Nano Vesicles) ⅓×(0.5 μg/ml), 1/15×(0.1 μg/ml) and 1×(1.5 μg/ml)” groups treated with artificial nanosomes derived from human umbilical cord-derived mesenchymal stem cells according to concentration along with elastase, and H&E staining was performed thereon.

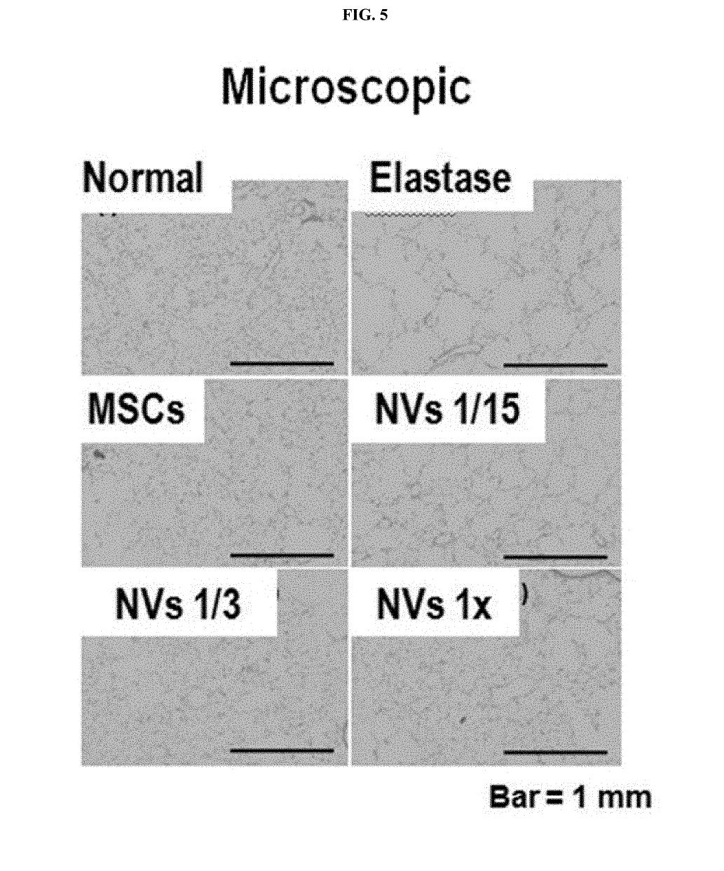

As a result, as illustrated in FIG. 5, it was confirmed that a degree of H&E staining was low in the “MSCs” group since regeneration did not progress well in an alveolar tissue, while the degree of H&E staining was high in the “NVs 1×” group treated with the artificial nanosomes derived from human umbilical cord-derived mesenchymal stem cells at the highest concentration since the alveolar tissue was regenerated most. As such, the artificial nanosomes derived from human umbilical cord-derived mesenchymal stem cells exhibited a more excellent regeneration effect in a concentration-dependent manner than that of the mesenchymal stem cells.

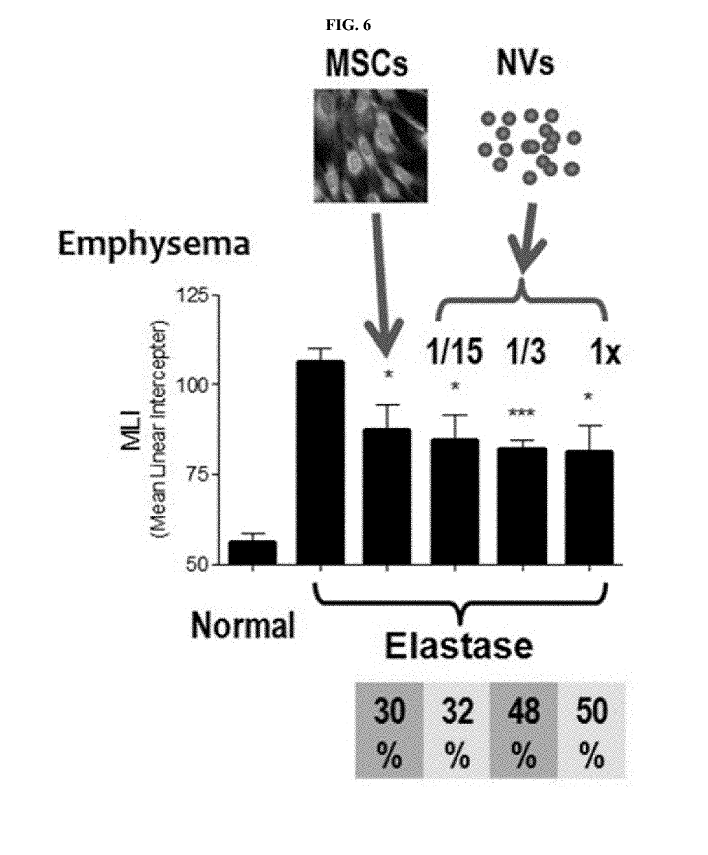

In addition, when MLI values, from which an alveolar regeneration rate was obtained, were measured, as illustrated in FIG. 6, the artificial nanosomes derived from human umbilical cord-derived mesenchymal stem cells exhibited a more excellent regeneration effect in a concentration dependent manner than that of the mesenchymal stem cells.

From the above results, it can be seen that artificial nanosomes derived from umbilical cord-derived mesenchymal stem cells exhibit an excellent alveolar regeneration effect.

### Example 7. Verification of Increase in Expression of Growth Factors of Umbilical Cord Mesenchymal Stem Cell-Derived Artificial Nanosomes

To measure changes in expression amounts of VEGF, FGF2, and HGF when a damaged pulmonary tissue of the elastase-induced emphysema animal model produced using the method of Example 2 was treated with each of artificial nanosomes derived from human umbilical cord-derived mesenchymal stem cells and mesenchymal stem cells, RIPA buffer (Cell Signaling Technology, Danvers, Mass.) was added to 10 mg of a pulmonary tissue extracted from the animal model, followed by homogenization, and protein quantitation was performed thereon using the Brad-ford assay. At this time, VEGF, FGF-2, and HGF levels were measured by ELISA according to the protocol of R&D systems (Minneapolis; MN).

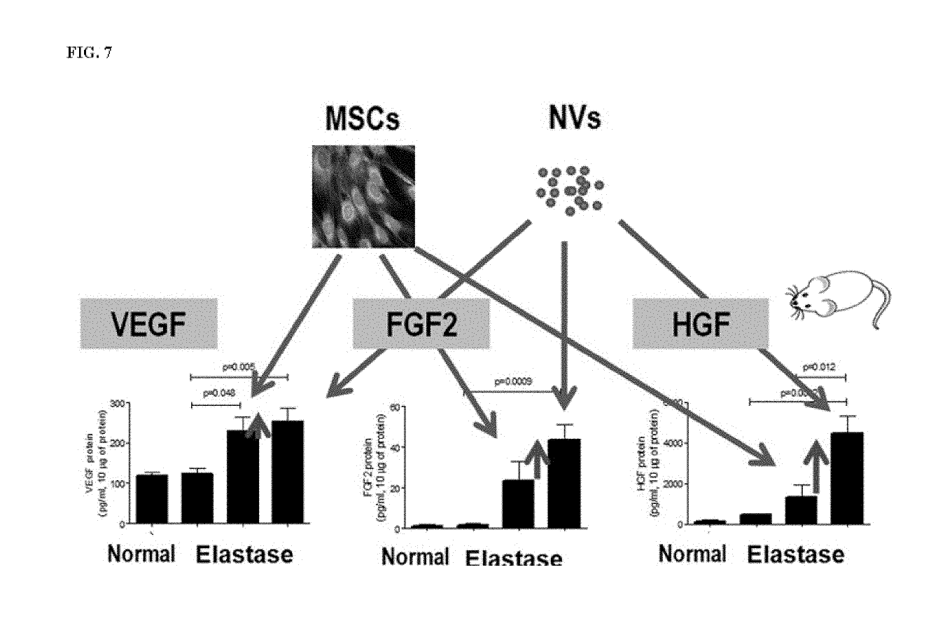

As a result, as illustrated in FIG. 7, it was confirmed that expression amounts of VEGF, FGF2, and HGF were significantly increased in a group treated with both elastase and the artificial nanosomes derived from human umbilical cord-derived mesenchymal stem cells compared to a group treated with both elastase and mesenchymal stem cells.

This indicates that the artificial nanosomes derived from human umbilical cord-derived mesenchymal stem cells have a potential capable of effectively inducing the regeneration of alveoli destructed by emphysema.

As is apparent from the foregoing description, mesenchymal stem cell-derived artificial nanosomes of the present disclosure exhibit a significantly excellent capability to regenerate alveoli compared to mesenchymal stem cells themselves and mesenchymal stem cell-derived natural exosomes, and thus can be effectively used for the treatment of a variety of pulmonary diseases including emphysema. In addition, the mesenchymal stem cell-derived artificial nanosomes can be collected through a simple production method using an extruder and an ultracentrifuge, instead of treating mesenchymal stem cells with a separate chemical substance. Thus, the possibility of occurrence of side effects that are caused by stem cell therapeutic agents is low and problems in terms of treatment efficiency pointed out as a drawback when mesenchymal stem cells are treated can be addressed. Accordingly, the mesenchymal stem cell-derived artificial nanosomes are expected to be effectively used as a therapeutic agent for the prevention or treatment of a pulmonary disease and for pulmonary cells.

The foregoing description of the present disclosure is provided for illustrative purposes only, and it will be understood by those of ordinary skill in the art to which the present invention pertains that the present disclosure may be easily modified in other particular forms without changing the technical spirit or essential characteristics of the present invention. Thus, the embodiments described herein should be construed as being provided for illustrative purposes only and not for purposes of limitation.

1. A method of treating a pulmonary diseases in a subject in need thereof comprising administering an effective amount of artificial nanosomes derived from mesenchymal stem cells to the subject.

2. The method of claim 1, wherein the artificial nanosomes derived from mesenchymal stem cells induce regeneration of alveoli.

3. The method of claim 1, wherein the mesenchymal stem cells are mesenchymal stem cells derived from fat, umbilical cord, umbilical cord blood, or bone marrow.

4. The method of claim 1, wherein the artificial nanosomes derived from mesenchymal stem cells are produced using an extruder and an ultracentrifuge.

5. The method of claim 1, wherein the pulmonary disease comprises at least one selected from the group consisting of emphysema, chronic bronchitis, pneumonia, pulmonary hypertension, chronic obstructive pulmonary disease, and asthma.

6. The method of claim 5, wherein the emphysema comprises at least one selected from the group consisting of panlobular emphysema, centrilobular emphysema, interstitial emphysema, and compensatory emphysema.

7. The method of claim 1, wherein the artificial nanosomes derived from mesenchymal stem cells increase expression of a growth factor.
